(19) United States
(12) Patent Application Publication
(10) Pub. No.: US 2011/0141418 A1
(43) Pub. Date: Jun. 16, 2011

(54) LIQUID CRYSTAL COMPOUNDS

(76) Inventors: Joanne E. Ennis, York (GB); John W. Goodby, York (GB); Rachel P. Tuffin, Worcestershire (GB); Ian C. Sage, Worcestershire (GB)

(21) Appl. No.: 13/056,924

(22) PCT Filed: Jul. 29, 2009
(86) PCT No.: PCT/GB09/01858

§ 371 Date: Jan. 31, 2011

(30) Foreign Application Priority Data
Jul. 30, 2008 (GB) 0813840.6

(51) Int. Cl. G02F 1/1337; C07C 321/00; C07C 69/74

(52) U.S. Cl. 349/123; 568/52; 560/1; 560/118

(57) ABSTRACT

The present invention relates to improved liquid crystal compounds which contain a mesogenic core which comprises a group of sub-formula (i) wherein R³ and R⁴ are independently selected from hydrogen, halogen or CF₃, provided at least one of R³ or R⁴ is selected from halogen or CF₃. The sub-formula (i) group may be located at any position within the mesogenic core of the liquid crystal compound, either at the terminus of the liquid crystal core or alternatively substantially in the middle of the liquid crystal core. The compounds of the invention provide compounds which when added to LC mixtures provide increasing birefringence, lowering of melting points, lowering clearing points, and lowering viscosities. These compounds and mixtures may fmd particular use in imaging or display media, such as monitors or televisions.

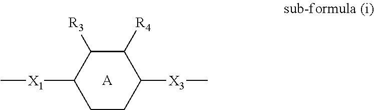

The present invention relates to novel compounds which are useful in the context of liquid crystal devices, either as liquid crystal compounds or as components of liquid crystal mixtures. The present invention also relates to processes for preparing such novel compounds and to liquid crystal mixtures or devices containing such compounds.

The phrase “liquid crystals” is well known. It refers to compounds which, as a result of their structure, have a phase or phases intermediate between liquid and solid and which are characterised by orientational ordering and a decrease in positional ordering, preferably at working temperatures for example, of from −40 to 200° C. These materials are useful in various devices, in particular in liquid crystal display devices.

Liquid crystals can exist in various phases. In essence there are three different classes of liquid crystalline material, each possessing a characteristic molecular arrangement. Those classes are nematic, chiral nematic (cholesteric) and smectic. For a fuller description of liquid crystal phases and devices see for example “The Handbook of Liquid Crystals”, Ed. D Demus, J Goodby, G W Gray, H-W Spiess, V Vill, Pub. WileyVCH, 1998.

Broadly speaking, the molecules of nematic compounds will align themselves in a particular orientation in a bulk material. Smectic materials, in addition to being orientated in a similar way, will align themselves closely in layers.

A wide range of smectic phases exists for example, smectic A and smectic C. In the former, the molecules are aligned perpendicularly to a base or support, whilst in the latter, molecules may be inclined to the support. Some liquid crystal materials possess a number of liquid crystal phases upon varying the temperature. Others have just one phase. For example, a liquid crystal material may show the following phases on being cooled from the isotropic phase:—isotropic—nematic—smectic A—smectic C—solid. If a material is described as being smectic A then it means that the material possesses a smectic A phase over a useful working temperature range.

Such materials are useful, in particular, in display devices, where their ability to align themselves and to change their alignment under the influence of voltage is used to impact on the path of polarised light, thus giving rise to liquid crystal displays. These are widely used in devices such as watches, calculators, display boards or hoardings, televisions and computer screens, in particular, laptop computer screens etc. Several properties of the compounds impact on the speed with which the compounds respond to voltage charges, including molecule size, conductivity, viscosity, dielectric anisotropy (Δε) or dipole moment (μ) and, in the smectic C phase, the spontaneous polarisation, etc. Alternatively, the light may be unpolarised and a dichroic dye may be incorporated into the mixture to give a change in the optical properties on switching of the device (Guest-host LCD).

The properties of these compounds vary depending upon their structure. Therefore, compounds with different structures are useful in a liquid crystal mixture to establish a wide range of different properties which can then be specifically matched to the target application. For example, compounds with low birefringence of 0.12, such as some phenylcyclohexyl derivatives, have practical application in devices that use a reflective light mode of operation, whereas mixtures with a high birefringence allow the use of much thinner devices or transmissive mode.

According to a first aspect of the invention there is provided a liquid crystal compound of Formula (I) with a mesogenic core which comprises at least one group of sub-formula (i)

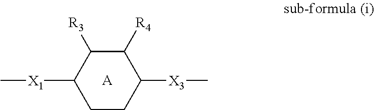

wherein R³ and R⁴ are independently selected from hydrogen, halogen or CF₃, provided at least one of R³ or R⁴ is selected from halogen or CF₃;

A is a 1,4,-carbocyclic aromatic ring or a fused carbocyclic aromatic ring, which may be optionally substituted;

X¹, X³ are linking groups independently selected from a direct bond, —S—, —SC(O)——OC(S)—, —SC(S)—, —CH₂CH₂—, —(CH₂)₄—, —CH₂O—, —CH═CH—, —C≡C—, —COO—, —OCO—, or —OCH₂—, provided that at least one of X¹ or X³, is selected from —S— or —SC(O)—. Preferably at least one of X¹ or X³, is selected from —S—.

Preferably the sulphur atom is located vicinal to the halogen or CF₃ moiety. When X¹ or X³ comprises a sulphur atom which is incorporated into a moiety that forms a linkage, such as, for example, —SC(O)— or —SC(S)—, preferably the sulphur atom part of the linkage is directly attached to the ring, such that said sulphur atom it is located vicinal to the R³ or R⁴ group. The thio-ester —SC(O)—, may, depending on which ring bears the halogen or CF₃ moiety, be considered as being orientated as —SC(O)— or —(O)CS—. Preferably, at least one of X¹ or X³ is selected from sulphur and the other is selected from oxygen. More preferably both X¹ and X³ aresulphur.

Preferably, if X¹ is selected from —S— or —SC(O)—, at least R³ is selected from halogen or CF₃ group, preferably fluorine. In a further embodiment where X³ is selected from —S— or —SC(O)— at least R⁴ is selected from halogen or CF₃ group, preferably fluorine.

In a further preferred embodiment, R³ and R⁴ are both selected from halogen or CF₃, preferably both are fluorine and at least one of X¹ or X³ is selected from sulphur. More preferably the other of X¹ or X³ is selected from oxygen or sulphur.

In a preferred embodiment the group of sub-formula (i) is a group of sub-formula (ii)

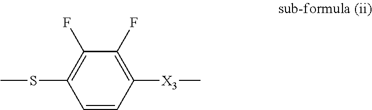

wherein X³ is as hereinbefore defined. Preferably, X³ is CH₂CH₂, or is a moiety which further increases the conjugation between the linking groups and the fluorine atoms, such as, for example, a moiety comprising an oxygen or sulphur atom, such as, for example, —O—, —COO—, —OCO—, —S—, more preferably X³ is —O— or —S—.

It is within the scope of the invention that the group of sub-formula (i) or sub-formula (ii) may be located at any position within the mesogenic core of the liquid crystal compound, that is to say it may be located at a terminal position at either end of the liquid crystal core or alternatively substantially in the middle of the liquid crystal core. There may also be one or more groups of sub-formula (i) or sub-formula (ii) present in the mesogenic core of the liquid crystal compound.

The mesogenic core of Formula I may possess any known 5 or 6-membered rings that are commonly used in liquid crystal mesogenic cores, provided at least one of the rings is of sub-formula (i) or (ii).

In a further embodiment there is provided a compound of Formula (II)

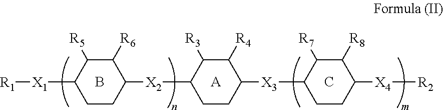

wherein R¹ and R² are any commonly used terminal end groups, preferably they are independently selected from cyano, halo, a functional group, optionally substituted hydrocarbyl, optionally substituted alkoxy, optionally substituted heterocyclyl, a group R¹³C(O)O— or R¹³OC(O)— where R¹³ is optionally substituted hydrocarbyl;

R³, R⁴ are as defined hereinbefore, R⁵, R⁶, R⁷ and R⁸ are independently selected from hydrogen, halogen, cyano or CF₃ provided at least one of R³, R⁴, R⁵, R⁶, R⁷ and R⁸ is selected from halogen or CF₃⁻;

X¹, X³, are as defined hereinbefore, X² and X⁴ are independently selected from a direct bond, —S—, —SC(O)——OC(S)—, SC(S)—, —CH₂CH₂—, —(CH₂)₄—, —CH₂O—, —CH═CH—, C≡C—, —COO—, —OCO—, —O— or —OCH₂—, provided that at least one of X¹, X², X³ andX⁴ is selected from —S— or —SC(O);

A is defined as hereinbefore, B and C are independently selected from carbocyclic aromatic ring, a fused carbocyclic aromatic ring or a heterocyclic ring, any of which may be optionally substituted;

and n is 0, 1 or 2, m is 0 or 1, provided that m+n is 1 or 2, further provided that the at least one —S— or —SC(O)— group is vicinal to at least one halogen or CF₃.

Preferably the halogen is fluorine. Preferably the sulphur linkage is —S—.

More preferably B and C are independently selected from a 1,4-phenylene, 1,4-cyclohexyl or a heterocyclic ring, any of which may be optionally substituted.

This vicinal arrangement of the sulphur atom to the halogen or CF₃ may be satisfied by;

i) when n=0 and m=1, at least one of X¹ or X³ is selected from —S— or —SC(O)— and at least one of R³ or R⁴ is selected from halogen or CF₃, or

ii) when n=1 and m=1,

either, at least one of X¹ or X² is selected from —S— or —SC(O)— and at least one of R⁵ or R⁶ is selected from halogen or CF₃, or

at least one of X³ or X⁴ is selected from —S— or —SC(O)— and at least one of R⁷ or R⁸ is selected from halogen or CF₃, or

at least one of X² or X³ is selected from —S— or —SC(O) and at least one of R³ or R⁴ is selected from halogen or CF₃.

At least one of the linking groups X¹, X², X³or X⁴ is independently selected from a sulphur containing linkage, such as, for example, —S— or —SC(O)— and is attached to a ring which contains a halogen or CF₃ substituent that is preferably in a vicinal position to said sulphur linkage, preferably the halogen is fluorine.

The sulphur linkage may be located in a terminal position i.e. such that the sulphur forms part of the terminal end group. This may arise in a 2 ring system when n=0 and m=1, X¹ is selected from —S— or —SC(O)— and at least R³ is selected from halogen or CF₃, preferably fluorine, more preferably both R³ and R⁴ are fluorine.

Alternatively, the sulphur linkage may be located in a non-terminal position, this may arise in a 2 ring system, such as, for example, when n=0 and m=1, X³ is selected from —S— or —SC(O)— and at least R⁴ is selected from halogen or CF₃, preferably fluorine, more preferably both R³ and R⁴ are fluorine.

In a 3-ring system, the sulphur linkage may be located in a terminal position, such as, for example, when n=1 and m=1, X¹ is selected from —S— or —SC(O)— at least R⁵ is selected from fluorine, preferably both R⁵ and R⁶ are fluorine; or X⁴ is selected from —S— or —SC(O)— and at least R⁸ is selected from fluorine, preferably both R⁷ and R⁸ are fluorine.

Alternatively in a 3 ring system, the sulphur may be located in a non-terminal position, such as, for example, when n=1 and m=1, X³ is selected from —S— or —SC(O)— and at least R⁷ is selected from fluorine; or X² is selected from —S— or —SC(O)— and at least R⁶ is selected from fluorine. Preferably, in either case, both R⁵ and R⁶ are fluorine;

Preferably when n=1 and m=1, X² is selected from —S— or —SC(O) and at least R³ is selected from fluorine; or X³ is selected from —S— or —SC(O) and at least R⁴ is selected from fluorine, preferably both R³ and R⁴ are fluorine.

In a further preferred embodiment, the sulphur linkage may connect two rings, where each of the linked ring may contain a halogen or CF₃ which is located in a position which is vicinal to said sulphur containing linkage, such as, for example, if X² is present and is selected from —S—, R³ and R⁶ may both be halogen or CF₃, preferably the halogen is fluorine.

As used herein, the term “hydrocarbyl” refers to any structure comprising carbon and hydrogen atoms. For example, these may be alkyl, alkenyl, alkynyl, aryl such as phenyl or naphthyl, arylalkyl, cycloalkyl, cycloalkenyl or cycloalkynyl. Suitably they will contain up to 20 and preferably up to 10 carbon atoms.

The term “heterocyclic” includes aromatic or non-aromatic rings, for example containing from 4 to 20, suitably from 5 to 10 ring atoms, at least one of which is a heteroatom such as oxygen, sulphur or nitrogen. Examples of such groups include furyl, thienyl, pyrrolyl, pyrrolidinyl, imidazolyl, triazolyl, thiazolyl, tetrazolyl, oxazolyl, isoxazolyl, pyrazolyl, pyridyl, pyrimidinyl, pyrazinyl, pyridazinyl, triazinyl, quinolinyl, iosquinolinyl, quinoxalinyl, benzthiazolyl, benzoxazolyl, benzothienyl or benzofuryl.

As used herein, the term “alkyl” refers to straight or branched chain alkyl groups, suitably containing up to 20 and preferably up to 6 carbon atoms, and the term “alkoxy” relates to —O-alkyl groups. The term “alkenyl” and “alkynyl” refer to unsaturated straight or branched chains which include for example from 2-20 carbon atoms, for example from 2 to 6 carbon atoms. In addition, the term “aryl” refers to aromatic groups such as phenyl or naphthyl. The terms “cycloalkyl”, “cycloalkenyl” and “cycloalkynyl” refer to such groups which are cyclic and have at least 3 and suitably from 5 to 20 ring atoms. These rings may be fused together to form bicyclic, tricyclic or even larger multiple ring systems.

Optionally substituted hydrocarbyl groups may be substituted by functional groups, or by other types of hydrocarbyl group. For example, cyclic groups such as aryl, heterocyclic or cycloalkyl, cycloalkenyl or cycloalkynyl, any of which may be substituted by hydrocarbyl chains such as alkyl, alkenyl or alkynyl groups as well as functional groups. Where the hydrocarbyl group is itself an alkyl, alkenyl or alkynyl group, it may be substituted with cyclic groups such as heterocyclic groups, aryl groups, cycloalkyl, cycloalkenyl or cycloalkynyl groups, as described above, which may themselves be further substituted by hydrocarbyl or functional groups. Optionally substituted hydrocarbyl may also have one or more non-adjacent carbon atoms replaced by O, S, CO₂, or OCO or —C≡C—.

The term “functional group” refers to reactive groups such as halo, cyano, nitro, oxo, C(O)OR^{a}, C(O)R^{a}, OC(O)R^{a}, OR^{a}, S(O)ₜR^{a}, NR^{b}R^{c}, OC(O)NR^{b}R^{c}, C(O)NR^{b}R^{c}, —NR^{b}C(O)OR^{a}, —NR^{b}C(O)R^{a}, —NR^{a}CONR^{b}R^{c}, ═NOR^{a}, —N═CR^{b}R^{c}, S(O)ₜNR^{b}R^{c} or —NR^{b}S(O)ₜR^{a} where R^{a}, R^{b} and R^{c} are independently selected from hydrogen or optionally substituted hydrocarbyl, or R^{b} and R^{c} together form an optionally substituted ring which optionally contains further heteroatoms such as sulphur, S(O),S(O)₂, oxygen and nitrogen, t is 0 or an integer of from 1-3.

The term “heteroatom” as used herein refers to non-carbon atoms such as oxygen, nitrogen, selenium or sulphur atoms as mentioned above. Where nitrogen atoms are present, they may be present as part of an amino residue such that they will be substituted for example by hydrogen or alkyl.

Conveniently, when present hydrocarbyl groups may be substituted by alkyl, alkoxy or halogen.

In the above defined liquid crystal compound, groups R¹ and R² represent suitable terminal end groups, while the remaining interposed structure represents the mesogenic core.

Usually R¹ and R² will not comprise further ring systems (to those of A, B, or C); in particular R¹ and R² will not usually be selected from an optionally substituted 1,4-phenylene, 1,4-cyclohexyl or a heterocyclic ring.

When R¹ and R² are alkyl or alkoxy groups, they suitably have from 3 to 8 carbon atoms, and preferably have from 3 to 5 carbon atoms. Suitably these carbon atoms are arranged in a straight chain.

In a preferred embodiment ring A is 1,4-phenylene or naphthyl and preferably rings B and C, when present, are selected from 1,4-phenylene, 1,4-cyclohexyl, 2,5-dioxanyl, pyridyl or 2,5-pyrimidinyl. Preferably B and C are 1,4-phenylene or 1,4-cyclohexyl.

Preferred optional substituents for rings A, B and C, are halogen and in particular fluorine, and advantageously, all substituents, when present, on these rings are fluorine.

Preferably, at least one ring of A, B and C, when present, includes two fluorine substituents arranged on adjacent carbon atoms within that ring. Preferably all fluorine atoms which are present are on the same side of the structure.

The use of lateral fluorine-substitution of the rings imparts strong lateral dipolar properties, resulting in the materials exhibiting negative dielectric anisotropy in the nematic phase. It is known that the incorporation of fluorine substituents, usually at least three fluorine substituents, in a mesogenic core may provide a particularly strong negative dielectric anisotropy and in the smectic C phase increases the dielectric biaxiality. In the invention the fluorine substituents may be present on any one of rings A, B or C, preferably at least one fluorine group is present on a ring which has at least one sulphur containing linkage. Any cyclohexyl rings present are preferably not substituted; however, if the cyclohexyl rings are substituted with at least one fluorine atom, care must be taken to avoid the loss of hydrogen fluoride.

The appropriate selection of the degree of fluorination or electron withdrawing groups on the ring containing the sulphur linkage results in compounds of Formula (I) or Formula (II), which may provide liquid crystals which are suitable for use in a number of modes such as: positive dielectric anisotropy nematics (AM/TN/STN), negative dielectric anisotropy nematics(VA mode) and smectics, such as ferroelectric, antiferroelectric and electroclinic devices. The materials of the invention may be able to align in the nematic phase in either homeotropic or planar orientation as required, depending on the surface treatment of the device.

The advantage of locating a sulphur atom at a vicinal location to the electron withdrawing group or groups, i.e. halogen or CF₃, particularly fluorine, is that it increases the electron withdrawing of the electron withdrawing group, which in turn provides an increase in the dipole moment across the ring, in the mesogenic core. An increase in dipole leads to the advantages of improved switching speed for a given voltage and/or allows a lower voltage to be used for a given speed, i.e. reduced voltage operation of devices incorporating the material with respect to devices without the material.

Preferably, ring A is a 1,2-difluorophenylene unit, such that at least one fluorine atom is located adjacent to a sulphur atom to permit conjugation and dipole alignment to take place. This arrangement has advantageously been found to generate strong negative dielectric anisotropy in liquid crystal compounds and mixtures. The increase in dipole may be further enhanced by selecting the other linking group from —O— or —S-.

A further advantage is that the sulphur is relatively facile to incorporate into part of a linkage, either as part of a thio-ester or a direct sulphur linkage i.e. thio-ether. A yet further advantage is that when the electron withdrawing group is fluorine, the lone pair of the sulphur atom and the lone pair of the fluorine are co-aligned this further increases the electron withdrawing effect of the flourine. This leads to advantages of increased dipole and polarisability in the liquid crystalline compound.

It has advantageously been found that the incorporation of a sulphur linkage and a halogen group on the same ring leads to higher birefringence, lower melting points, lower clearing points and/or lower viscosities when compared to their oxy- or carbon-substituted equivalents. The presence of a lower viscosity is highly unexpected as larger, more sterically hindered atom, would be expected to increase the viscosity.

Single liquid crystal materials are unlikely to show all the properties required of the liquid crystal material present in a device thus mixtures comprising one or more compounds of

Formula (I), Formula (II), or mixing with other known liquid crystal compounds, may be necessary to achieve the desired results. It is important that the compounds remain in solution with each other; this is a particular problem in smectic C mixtures.

In a preferred embodiment of the invention, the group of sub-formula (i) is present in a central position in the mesogenic core. Accordingly, there is provided a compound of Formula (III)

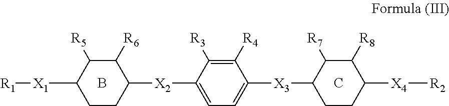

wherein R¹, R², R⁵, R⁶, R⁷, R⁸, X¹, X⁴, B and C are as defined hereinbefore, R³ and R⁴ are halogen and at least one of X² or X³ is —S—. Preferably R³ and R⁴ are both fluorine. Preferably at least one of X² or X³ is —S— and the other is —O— or —S—.

In a further embodiment of the invention, the group of sub-formula (i) is present in a terminal position in the mesogenic core, providing a compound of Formula (IV)

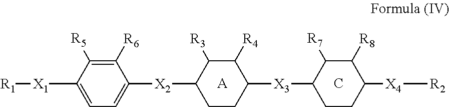

wherein R¹, R², R³, R⁴, R⁷, R⁸, X³, X⁴, A and C are as defined hereinbefore, R⁵ and R⁶ are halogen and at least one of X¹ or X² is —S—. Preferably R⁵ and R⁶ are both fluorine. Preferably at least one of X¹ or X² is —S— and the other is —O— or —S—.

In a further embodiment of the invention, the group of sub-formula (i) is present in a terminal position in the mesogenic core, providing a compound of Formula (V)

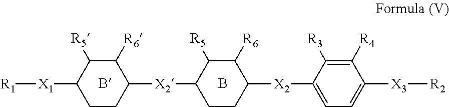

wherein R¹, R²R³, R⁴, X¹, X², X³, B is as defined hereinbefore, B′ is selected from B, X^{′} is selected from X², R^{5′} and R^{6′} are selected as R⁵ and R⁶ respectively, R³ and R⁴ are halogen and at least one of X³ or X² is —S—. Preferably R³ and R⁴ are both fluorine. Preferably at least one of X³ or X² is —S— and the other is —O— or —S—.

Typically most liquid crystalline compounds contain 3 rings as part of the mesogenic core. However, compounds which posses only 2 rings as part of the mesogenic core are often provided as dopants or components of an overall liquid crystal mixture. There is further provided a compound of Formula (VI),

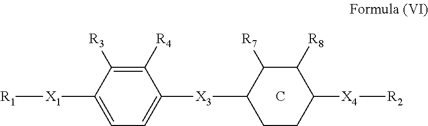

wherein R¹, R², R⁷, R⁸, X⁴, and C are as defined hereinbefore, R³ and R⁴ are halogen and at least one of X¹ or X³ is —S—. Preferably R³ and R⁴ are fluorine. Preferably at least one of X¹ or X³ is —S— and the other is —O— or —S—, more preferably —O—.

According to a further aspect of the invention a process for preparing a compound of sub-formula (i) or inserting said groups of sub-formula (i) into a compound of Formula (I), (II), (Ill), (IV), (V) or (VI). There is further provided a process for preparing a compound of Formula (I) or (II).

In a further aspect, the invention provides a liquid crystal mixture comprising at least one compound as described above. Suitably, a liquid crystal mixture may comprise at least two different compounds according to the invention, which may be independently selected from compounds of Formula (I), (II), (Ill), (IV), (V) or (VI), and optionally other liquid crystal compounds.

In a further aspect of the invention there is provided the use of a compound according to the invention as a liquid crystal compound.

There is further provided a method of forming a liquid crystal device, said method comprising selecting a starting material which comprises a compound of Formula (I), (II), (III), (IV), (V) or (VI), and incorporating it in a liquid crystal device.

Compounds of the invention may have application in liquid crystal devices, and one convenient mode is the use in a reflective light mode of operation. They may also be suitable for applications in liquid crystal on silicon (LcoS) devices and also in twisted nematic(TN) (for positive dielectric anisotropy materials) and vertically aligned nematic (VAN) devices (for negative dielectric anisotropy materials). In addition, they may be useful in ferroelectric displays and in super twisted nematics(STN), Active Matrix, or TN devices operating with positive dielectric anisotropy.

The invention also provides a liquid crystal device comprising at least one compound of Formula (I), (II), (Ill), (IV), (V) or (VI), or a liquid crystal mixture as hereinbefore defined.

A further aspect of the invention provides a method of increasing birefringence, lowering melting points, lower clearing points, and lowering viscosities, comprising the use of at least one compound of Formula (I), (II), (Ill), (IV), (V) or (VI), or a liquid crystal mixture as hereinbefore defined.

A further aspect of the invention provides a device comprising two spaced cell walls each bearing electrode structures and treated on at least one facing surface with an alignment layer, a layer of a liquid crystal material enclosed between the cell walls, characterised in that it comprises at least one compound or a liquid crystal mixture according to the invention.

In an alternative arrangement a device contains cell walls which comprise at least 4 electrodes such as to allow said liquid crystal compound or mixture to be switched in more than one direction.

Further provided is a bistable nematic liquid crystal device comprising; two cell walls enclosing a layer of liquid crystal material or a mixture (as hereinbefore defined);

electrode structures on both walls;

a surface alignment on the facing surfaces of both cell walls providing alignment to liquid crystal molecules;

means for distinguishing between switched states of the liquid crystal material;

a surface alignment grating on at least one cell wall that permits the liquid crystal molecules to adopt two different pre-tilt angles in the same azimuthal plane;

the arrangement being such that two stable liquid crystal molecular configurations can exist after suitable electrical signals have been applied to the electrodes;

wherein the layer of liquid crystal material comprises a compound of Formula (I) or Formula (II).

The invention further provides a novel feature or any combination of novel features as identified above. In a further aspect, the invention provides any compound that is suitable for use as a liquid crystal compound and which has Formula (I) or Formula (II) as defined above, except that R¹ and R² may be replaced by any terminal end groups commonly used as end groups in liquid crystal compounds.

A number of compounds have been synthesised and are presented as a series of mesogenic cores with different linking groups.

2 Ring series

| Compound Structure | Series |
|---|---|
| 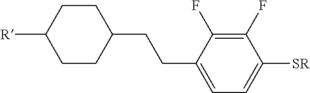 | A |
| 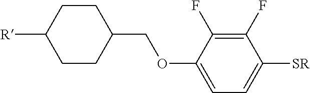 | B |
| 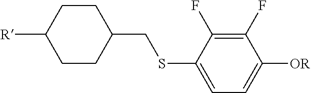 | C |
| 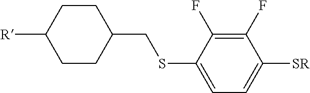 | D |

3 Ring Series

| Compound Structure | Series |
|---|---|
| 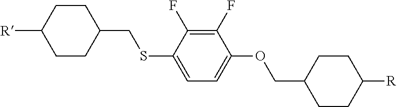 | E |
| 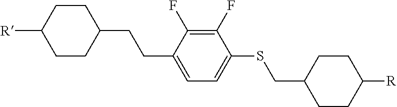 | F |
| 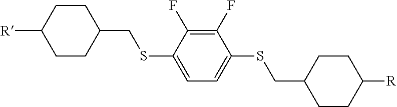 | G |
| 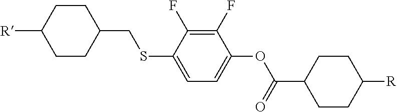 | H |
| 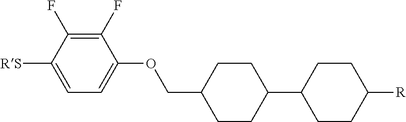 | I |
| 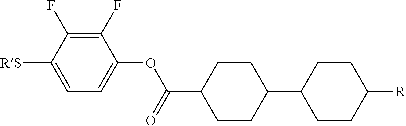 | J |

Synthesis of intermediate A

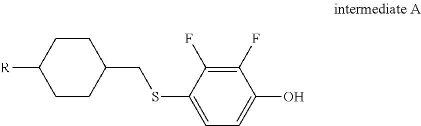

Intermediate A is a useful precursor compound for several of the above series, such as, for example, series C, D, E and G.

According to a further aspect of the invention there is provided a compound of intermediate A, where R may be selected from R¹ as defined hereinbefore, preferably hydrocarbyl.

The synthesis route to the thioether intermediate A compound trans-4-n-alkylyclohexylmethylenethio-2,3-difluorophenol is shown in Scheme 1, below.

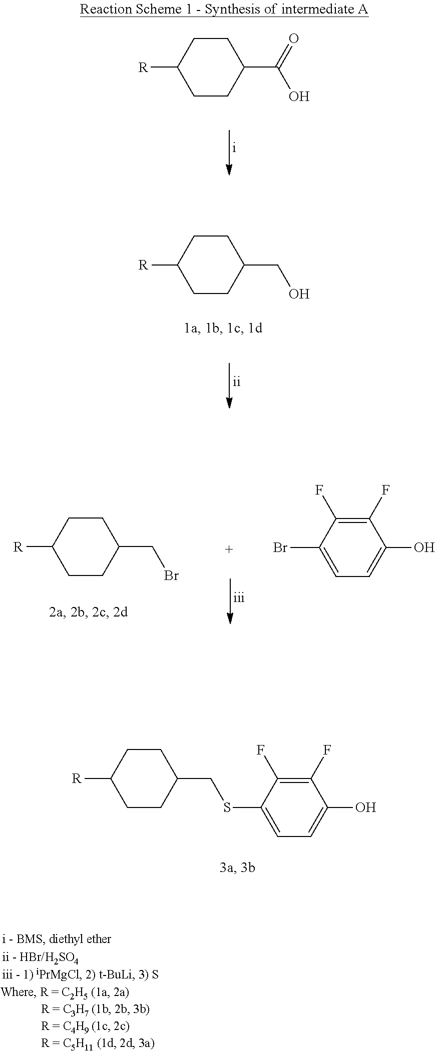

The appropriate trans-4-n-alkylcyclohexyl-1-methylbromide (2a, 2b, 2c, 2d) was synthesised from readily available trans-4-n-alkylcyclohexane carboxylic acids. The latter were reduced with borane-dimethylsulfide complex in anhydrous diethyl ether yielding the corresponding trans-4-n-alkylcyclohexyl-1-methanol (la, 1 b, 1 c, 1d). The methan-ol (1a, 1b, 1c, 1d) was subsequently heated under reflux in a mixture of H₂SO₄ and HBr to yield the desired methylbromide (2a, 2b, 2c, 2d).

Commercially available 4-bromo-2,3-difluorophenol was protected with isopropyl magnesium chloride at low temperature (0° C.) in tetrahydrofuran. After 1 hour, the reaction mixture was cooled to −78° C. and lithiation was carried out using tert-butyllithium. The reaction mixture was stirred at this temperature for 1 hour, before addition of sulfur powder. The reaction was subsequently stirred until it became pale yellow and then warmed to room temperature for 15 min before the addition of a trans-4-n-alkylcyclohexyl-1-methylbromide (2b, 2d). The product was purified by recrystallisation yielding the corresponding 2,3-difluoro-4-((trans-4-n-pentylcyclohexyl)methylenethio)phenol (3a, 3b).

The compounds according to the invention may be synthesised by any known pathways. Particularly preferred reaction schemes are shown for the E, H and J series and are detailed below.

Synthesis of Ether linked products (E-Series)

A series of ether linked compounds where R is ethyl, propyl, butyl or pentyl and R¹ is propyl or pentyl have been synthesised using, the reaction detailed in scheme 2, below.

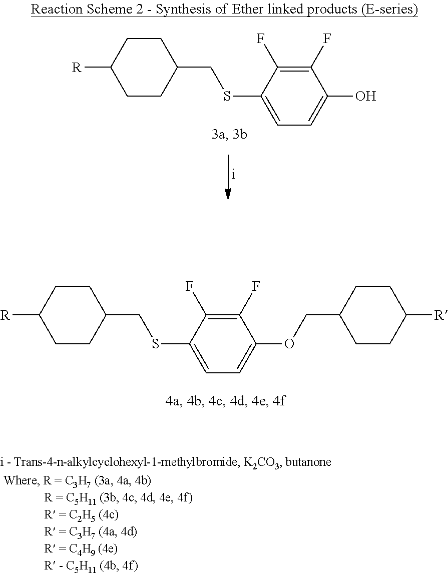

2,3-Difluoro-4-((trans-4-n-pentylcyclohexyl)methylenethio)phenol (3) was heated under reflux in butanone in the presence of trans-4-n-alkylcyclohexyl-1-methylbromide (2) and K₂CO₃. This yielded the desired trans-4-n-alkylcyclohexylmethyleneoxy-2,3-difluorophenyl trans-4-n-pentyl cyclohexylmethyl thioethers (4).

The ether:thioether linked compounds exhibit monotropic nematic and smectic A phases; the transition temperatures are given in Table 1 to 4. All of the compounds are white crystalline solids at room temperature.

Ether Series (E)

TABLE 1
Transition temperatures of Pentyl series

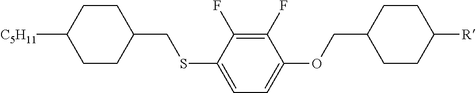

| Compound identifier | Chain Length | Transition Temperatures (° C.) |
|---|---|---|
| 5E2 | R′ = C₂H₅ | Mpt 66 |
| 5E3 | R′ = C₃H₇ | Cr 61 N (45) I |
| 5E4 | R′ = C₄H₉ | Cr 64 SmA (39) N (45) I |
| 5E5 | R′ = C₅H₁₁ | Cr 56.2 SmA (49.2) N (53.2) I |

TABLE 2
Transition temperatures of Butyl series

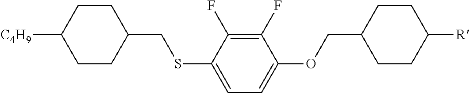

| Compound identifier | Chain Length | Transition Temperatures (° C.) |
|---|---|---|
| 4E2 | R′ = C₂H₅ | Mpt 54.4 |
| 4E3 | R′ = C₃H₇ | Cr 49.7 N (35.2) I |
| 4E4 | R′ = C₄H₉ | Mpt 47.0 |
| 4E5 | R′ = C₅H₁₁ | Cr 45.8 N (40.8) I |

TABLE 3
Transition temperatures of Propyl series

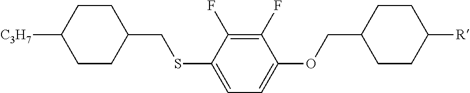

| Compound identifier | Chain Length | Transition Temperatures (° C.) |
|---|---|---|
| 3E2 | R′ = C₂H₅ | Mpt 46.6 |
| 3E3 | R′ = C₃H₇ | Cr 47.1 N (35.2) I |
| 3E5 | R′ = C₅H₁₁ | Cr 43.5 N (40) I |

TABLE 4
Comparison of effect of alkyl chain length on phase behaviour

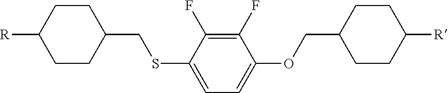

| Compound | | Chain | Transition |
|---|---|---|---|
| identifier | Chain length R | length R′ | Temperatures (° C.) |
| 3E3 | C₃H₇ | C₃H₇ | Cr 46.5 N (35) I |
| 5E3 | C₅H₁₁ | C₃H₇ | Cr 61 N (45) I |
| 3E5 | C₃H₇ | C₅H₁₁ | Cr 43.5 N (40) I |
| 5E5 | C₅H₁₁ | C₅H₁₁ | Cr 56.2 SmA (49.2) N (53.2) I |

Table 4, above shows a comparison of compound data from Tables 1 to 3 and shows that changing the length of the terminal alkyl chain attached to the thiother linked cyclohexyl group appears to have a greater effect on the transition temperatures than changing the terminal alkyl chain attached to the ether linked cyclohexyl group, as may be seen in Table 4.

The trend in transition temperatures is as expected for increasing the length of the alkyl side chains.

Synthesis of Ester Linked Products (H-Series)

A series of ester linked compounds where R is ethyl, propyl, butyl or pentyl and R is propyl or pentyl have been synthesised using the reaction shown in reaction scheme 3, below.

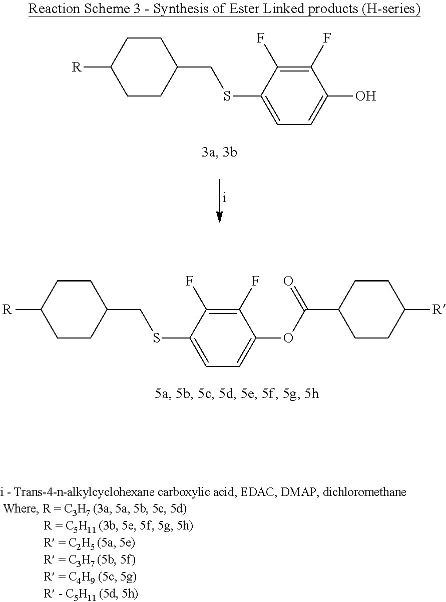

The ester compounds all exhibit a useful enantiotropic nematic temperature range. The ethyl compound 3H2, has a lower than expected melting point compared to the other compounds in the series. The transition temperatures are shown in Tables 5 to 7.

TABLE 5
Transition temperatures of the pentyl series

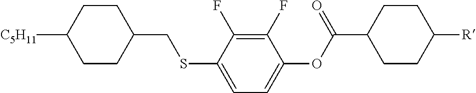

| | | Transition Temperatures |
|---|---|---|
| Compound identifier | Chain Length R′ | (° C.) |
| 5H2 | R′ = C₂H₅ | Cr 46.9 N 71.2 I |
| 5H3 | R′ = C₃H₇ | Cr 51.8 N 92.6 I |
| 5H4 | R′ = C₄H₉ | Cr 53.6 N 89.6 I |
| 5H5 | R′ = C₅H₁₁ | Cr 61.5 N 96.6 I |

TABLE 6
Transition temperatures of the butyl series

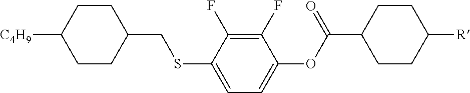

| | | Transition Temperatures |
|---|---|---|
| Compound identifier | Chain Length R′ | (° C.) |
| 4H2 | R′ = C₂H₅ | Cr 42.2 N 64.1 I |
| 4H3 | R′ = C₃H₇ | Cr 51.6 N 84.5 I |
| 4H4 | R′ = C₄H₉ | Cr 52.4 N 69.4 I |
| 4H5 | R′ = C₅H₁₁ | Cr 63.2 N 90.9 I |

TABLE 7
Transition temperatures of the propyl series

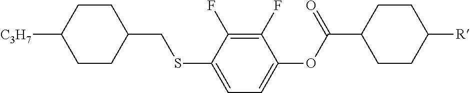

| | | Transition Temperatures |
|---|---|---|
| Compound identifier | Chain Length R′ | (° C.) |
| 3H2 | R′ = C₂H₅ | Cr 27.8 N 62.4 I |
| 3H3 | R′ = C₃H₇ | Cr 63.0 N 83.5 I |
| 3H4 | R′ = C₄H₉ | Cr 53.9 N 79.6 I |
| 3H5 | R′ = C₅H₁₁ | Cr 51.0 N 90.5 I |

Synthesis of bicyclohexyl ether Compounds (I-Series)

Several compounds of the I-series, bicyclohexyl ether compounds, have been synthesised, which were found to exhibit reasonably low melting points and wide nematic phase ranges.

TABLE 8
Transition temperatures of I series compounds

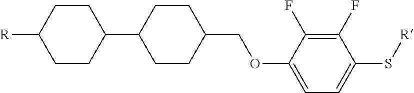

| Compound | Chain | Chain | Transition |
|---|---|---|---|
| identifier | Length R | Length R′ | Temperatures (° C.) |
| 3I3 | R = C₃H₇ | R′ = C₃H₇ | Cr 45.8 SmA (27.8) N 90.3 I |
| 3I4 | R = C₃H₇ | R′ = C₄H₉ | Cr 30.6 SmA (35.7) N 92.4 I |
| 3I5 | R = C₃H₇ | R′ = C₅H₁₁ | Cr 41.4 N 76.7 I |
| 5I3 | R = C₅H₁₁ | R′ = C₃H₇ | Cr 32.1 SmA 51.2 N 95.3 I |
| 5I4 | R = C₅H₁₁ | R′ = C₄H₉ | Cr 38.1 SmA 62.0 N 96.8 I |
| 5I5 | R = C₅H₁₁ | R′ = C₅H₁₁ | Cr 45.2 SmA 67.1 N 89.8 I |

Synthesis of bicyclohexyl ester Compounds (J-Series)

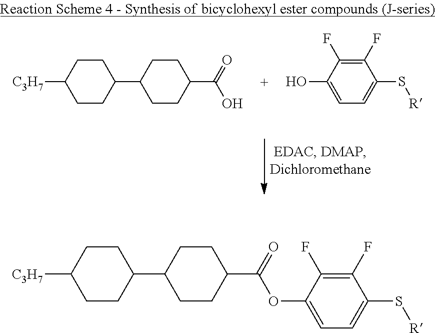

The bicyclohexyl ester compounds were synthesised from commercially available trans-4-n-(trans-4-n-alkylcyclohexyl)cyclohexane carboxylic acids in an analogous manner to the H-series compounds, in good yields.

The bicyclohexyl ester compounds exhibit nematic, smectic A and smectic B (hexatic) phases. The B phase stability increases with increasing length of the alkyl chain attached to the bicyclohexyl group, and decreases with increasing length of the alkyl chain attached to the thiodifluorophenyl group. The nematic clearing points show similar behaviour, most of the compounds also exhibit a smectic A phase, the stability of which is also dependant on alkyl chain length. The transition temperatures are shown in Tables 9-12.

TABLE 9
Transition temperatures of the ethyl series

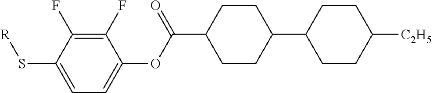

| Compound identifier | Chain Length R | Transition Temperatures (° C.) |
|---|---|---|
| 2J3 | R = C₃H₇ | Cr 36.3 SmB 88.6 N 124.9 I |
| 2J4 | R = C₄H₉ | Cr 40.4 SmB 72.0 SmA 93.2 N 131.1 I |
| 2J5 | R = C₅H₁₁ | Cr 53.8 SmB 72.9 SmA 95.9 N 122.1 I |

TABLE 10
Transition temperatures of the propyl series

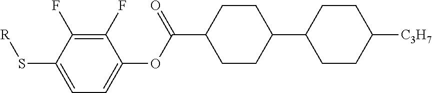

| Compound | | |
|---|---|---|
| identifier | Chain Length R | Transition Temperatures (° C.) |
| 3J3 | R = C₃H₇ | Cr 42.6 SmB 108.3 SmA 114.5 N 124.9 I |
| 3J4 | R = C₄H₉ | Cr 47.1 SmB 76.3 SmA 118.4 N 158.6 I |
| 3J5 | R = C₅H₁₁ | Cr 66.7 SmB 96.1 SmA 122.4 N 149.2 I |

TABLE 11
Transition temperatures of the butyl series

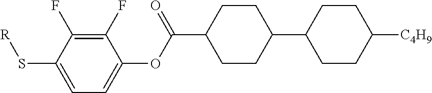

| Compound | | |
|---|---|---|
| identifier | Chain Length R | Transition Temperatures (° C.) |
| 4J3 | R = C₃H₇ | Cr 37.1 SmB 123.1 SmA 129.5 N 156.4 I |
| 4J4 | R = C₄H₉ | Cr 38.0 SmB 112.4 SmA 129.3 N 155.0 I |
| 4J5 | R = C₅H₁₁ | Cr 59.3 SmB 111.4 SmA 133.7 N 147.6 I |

TABLE 12
Transition temperatures of the pentyl series

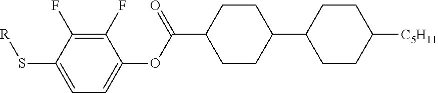

| Compound | | Transition |
|---|---|---|
| identifier | Chain Length R | Temperatures (° C.) |
| 5J3 | R = C₃H₇ | Cr 24.3 SmB 125.7 SmA 134.6 N 160.1 I |
| 5J4 | R = C₄H₉ | Cr 38.5 SmB 121.3 SmA 143.1 N 162.0 I |
| 5J5 | R = C₅H₁₁ | Cr 54.3 SmB 119.3 SmA 143.0 N 152.9 I |

Physical Properties of Mixtures

The properties of the two series of C₅ esters (H compounds) and C₅ ethers (5E compounds) were determined in mixtures, which were prepared in a neutral” dielectric anisotropy host compound 20-113 (obtained from Dai Nippon (DIG)). The properties of particular interest are transition temperatures, dielectric anisotropy, refractive indices and birefringence as a function of temperature, switching speed and rotational viscosity and stability to light and heat.

The physical properties of selected compounds were determined at 20% concentration in the dielectric neutral host system 20-113.

Mixtures—Phase Behaviour and Dielectric Anisotropy

The phase behaviour was determined by optical microscopy using a polarising microscope and a Mettler FP82 hot stage and controller. Refractive indices and birefringence were measured using an Abbé refractometer at various temperatures and the dielectric constants measured in planar (SiOₓ or Nissan SE130) and homeotropic (chrome complex) cells using a Hewlett Packard LCR meter at 25° C. The dielectric anisotropies of the compounds were extrapolated from that measured in the mixtures.

Tables 13 and 14 show the clearing points and dielectric anisotropy of the mixtures as well as the extrapolated dielectric anisotropy of the compounds. The esters, in general have higher magnitude of extrapolated dielectric anisotropy than the ether compounds, as well as higher temperature phase transitions in the mixtures.

TABLE 13
Clearing points and dielectric anisotropy of the mixtures and extrapolated
dielectric anisotropy

| | Compound, | | | | | |
|---|---|---|---|---|---|---|
| (20° C.) | (R) | % in 20-113 | Clearing point (° C.) | ε par | Δε mixture | Extrapolated Δε |
| Esters | 5H2 (C₂H₅) | 19.9 | 95.3-93.3 | 3.72 | −1.00 | −4.80 |
| (H) | 5H3 (C₃H₇) | 20 | 99.5-97.7 | 3.60 | −0.91 | −4.32 |
| C₅H₁₁ | 5H4 (C₄H₉) | 20.7 | 98.8-96.8 | 3.60 | −0.75 | −3.38 |
| | 5H5 (C₅H₁₁) | 20.6 | 100.5-98.5 | 3.61 | −0.74 | −3.33 |
| C₃H₇ | 3H3 (C₃H₇) | 19.26 | 99.0-97.1 | 3.48 | −0.78 | −4.04 |
| Host | 20-113 | 100 | 105.2-103.0 | 2.65 | −0.06 | N/A |

TABLE 14
Clearing points and dielectric anisotropy of the mixtures and extrapolated
dielectric anisotropy.

| | Compound, | | Clearing point | | Δε | Extrapolated |
|---|---|---|---|---|---|---|
| (20° C.) | (R) | % in 20-113 | (° C.) | ε par | mixture | Δε |
| Ethers (E) | 5E2 (C₂H₅) | 19.6 | 87.4-82.1 | 3.41 | −0.50 | −2.32 |
| C₅H₁₁ | 5E3 (C₃H₇) | 19.7 | 90.7-86.8 | 3.63 | −0.66 | −3.11 |
| | 5E4 (C₄H₉) | 19.4 | 91.1-85.0 | 3.69 | −0.70 | −3.35 |
| Host | 20-113 | 100 | 105.2-103.0 | 2.65 | −0.06 | N/A |

Comparison of Dielectric Anisotropy in a Negative Host

The dielectric anisotropy of negative materials has in the past been shown to be higher when measured in a negative host compared to the figures measured in positive and neutral hosts. A Merck negative dielectric mixture MLC6608 was used as a host to compare the measurement of dielectric anisotropy of 2 compounds. The results are shown in Table 15. The mixtures containing the thio ether and thio ester compound both exhibit higher dielectric anisotropy than the MLC6608 host; the extrapolated anisotropy is quite high for the ester compound at -7. Each compound also shows the anticipated increase in apparent negative anisotropy when measured in the negative host compared to a neutral one, amounting to an increase of 65-70%.

TABLE 15
Dielectric anisotropy measured by extrapolation in a negative host

| | Compound, | % in | Clearing point | | Δε | Extrapolated |
|---|---|---|---|---|---|---|
| (20° C.) | (R) | MLC6608 | (° C.) | ε par | mixture | Δε |
| host | MLC6608 | | 90.3-93.6 | 3.53 | −4.0 | — |
| ester | 5H3 (C₂H₅) | 15.47 | 91.7-88.9 | 3.20 | −4.50 | −7.2 |
| ether | 5E3 (C₃H₇) | 13.3 | 80.7-84.4 | 3.14 | −4.17 | −5.3 |

Refractive Index and Birefringence

Table 16 shows the refractive indices and birefringence at 25, 40 and 80° C. and at T_{N-I}-30° C. for the pentyl ester compound mixtures, while Table 17 gives the same data for the pentyl ether compound mixtures. The majority of the mixtures show the same birefringence, 0.083+/−0.002 at T_{N-I}-30° C., with the exception of the mixture containing 5H5 which has a lower birefringence of 0.078.

TABLE 16
Refractive indices and birefringence at various temperatures
for the ester (5H) series of compounds.

| | Temp | | | |
|---|---|---|---|---|
| | (° C.) | nₒ | nₑ | Δn |
| 5H2 | 80.0 | 1.5553 | 1.4809 | 0.074 |
| | 65.3 | 1.5666 | 1.4833 | 0.083 |
| | 40.0 | 1.5828 | 1.4899 | 0.093 |
| | 25.0 | 1.5899 | 1.4937 | 0.096 |
| 5H3 | 80.0 | 1.5581 | 1.4801 | 0.078 |
| | 69.5 | 1.5667 | 1.4823 | 0.084 |
| | 40.0 | 1.5840 | 1.4896 | 0.094 |
| | 25.0 | 1.5937 | 1.4940 | 0.100 |
| 5H4 | 80.0 | 1.5547 | 1.4800 | 0.075 |
| | 68.8 | 1.5642 | 1.4821 | 0.082 |
| | 40.0 | 1.5833 | 1.4901 | 0.093 |
| | 25.0 | 1.5921 | 1.4928 | 0.099 |
| 5H5 | 80.0 | 1.5516 | 1.4795 | 0.072 |
| | 70.5 | 1.5588 | 1.4804 | 0.078 |
| | 40.0 | 1.5801 | 1.4871 | 0.093 |
| | 25.0 | 1.5894 | 1.4930 | 0.096 |

TABLE 17
Refractive indices and birefringence at various
temperatures for the propyl ester (3H3) compound.

| Temp | | | |
|---|---|---|---|
| (° C.) | nₒ | nₑ | Δn |
| 20 | 1.5966 | 1.4960 | 0.1006 |
| 25 | 1.5951 | 1.4952 | 0.0998 |
| 40 | 1.5867 | 1.4907 | 0.0959 |
| 41.1 | 1.5824 | 1.4887 | 0.0937 |
| 50 | 1.5808 | 1.4878 | 0.0930 |
| 60 | 1.5742 | 1.4849 | 0.0893 |
| 67.1 | 1.5692 | 1.4839 | 0.085 |
| 70 | 1.5663 | 1.4828 | 0.0835 |

Comparison of Compounds B1 and C1

| | | |
|---|---|---|
| 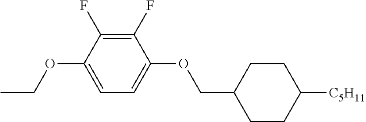 | K 38 (0-N) Iso. | Prior art |
| 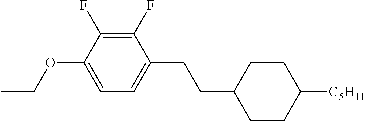 | K 49 (20 N) Iso | Prior art |
| 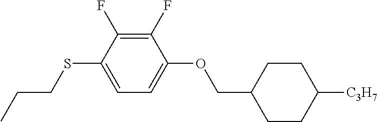 | K 21.2 [-58 N] Iso | B-series |
| 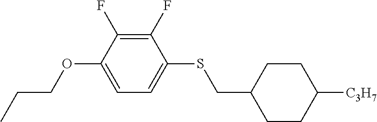 | K 5.0 [-55 N] Iso | C-series |

The B- and C- series show greatly reduced transition temperatures in comparison to above identified ethyl and ether linked prior art compounds. The presence of a sulphur atom which is vicinal to the diflurophenyl ring clearly reduces the transition temperature of the material, compared to a non-thio derivative.

Comparison of Compound 5E5

| | | |
|---|---|---|
| 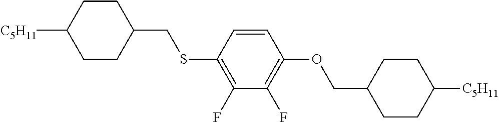 | 5E5 | E-series |
| K 56.2 SmA (49.2) N (53.2) I | | |
| 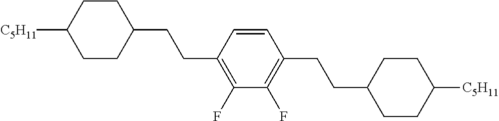 | a | Prior art |
| K 64 N 106.7 I | | |
| 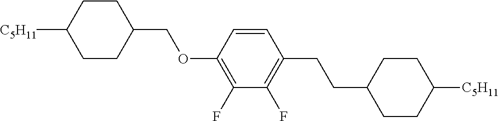 | b | Prior art |
| K76 (SmA 72) N 109.4 I | | |

For comparative purposes prior art compounds a and b were selected as they possess analogous core structure to the 5E5 and the same length alkyl chains. The 5E5 compound possess a sulphur atom which is vicinal to the difluorophenyl ring and it can be seen that the clearing point of 5E5 is reduced significantly compared to prior art compounds a and b.

Comparison of Compound 3I3

| | | |
|---|---|---|
| 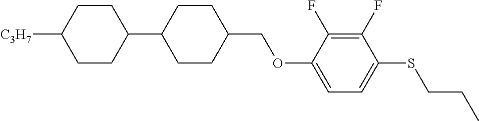 | 3I3 | I-series |
| K 45.8 (27.8 SmA) N 90.3 I | | |
| 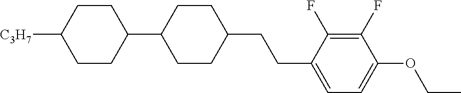 | c | Prior art |
| K 68 SmB 109 N 160 I | | |
| 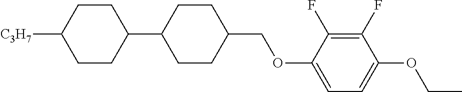 | d | Prior art |
| K 62 N 154 I | | |

The compound 3I3 (I-series compound) possesses a lower melting point than both of the prior art difluorophenyl derivates c and d, which are virtually identical in structure to compound 3I3. Furthermore, the I-series compound exhibits an unexpectedly lower clearing point, even though it has a slightly longer alkyl chain.

The invention will now be described by way of example only, with reference to the following Examples and drawings, in which:

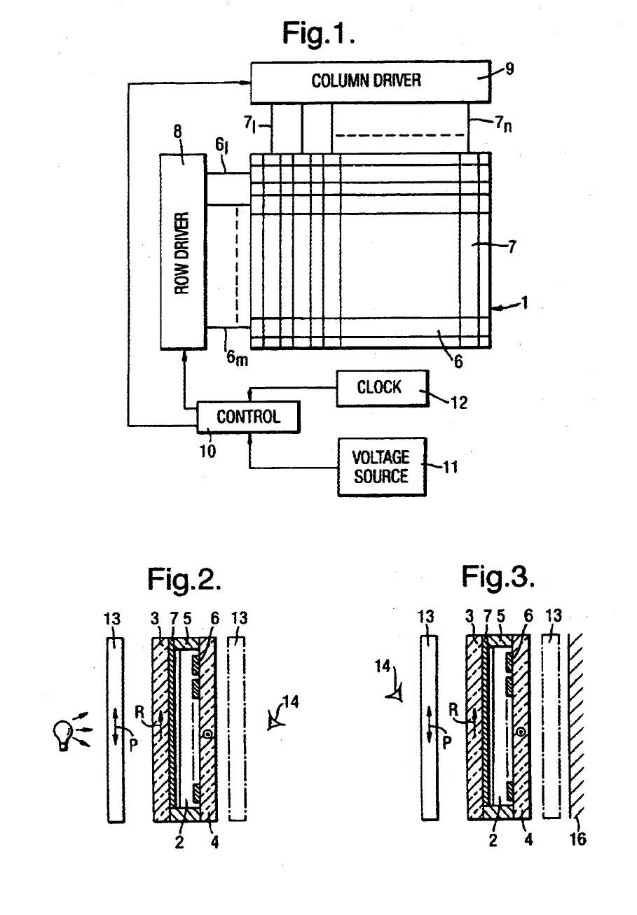

FIG. 1 is a plan view of a matrix multiplex addressed liquid crystal device; FIG. 2 is a cross-sectional view of the device of FIG. 1 operating in a transmissive mode, and;

FIG. 3 is similar to FIG. 2, but shows the device operating in a reflective mode.

The device of FIGS. 1, 2 and 3 comprises a liquid crystal cell 1 formed by a layer of a liquid crystal mixture 2 according to the invention contained between two glass walls 3, 4 spaced typically 1 to 15 μm apart by a spacer ring 5. The inside faces of both walls 3, 4 are coated with electrodes 6. The electrodes may be of sheet like form covering the complete wall, or formed into, for example, strip electrodes to provide an array of addressable electrode intersections. The walls are also coated with an aligning layer (not shown) of material according to the current invention.

If the mixture 2 is nematic, then the device may be the known super twisted nematic device, also known as a STN device. In this case, polarisers 13 are used to distinguish between the device voltage ON and OFF states.

The liquid crystal mixture may be a nematic, chiral nematic (cholesteric), or smectic (e.g., ferroelectric) mixture. The device may be used as a display device, e.g., displaying alpha numeric information, or an x, y matrix displaying information. Alternatively, the device may operate as a shutter to modulate light transmission, e.g. as a spatial light modulator, or as a privacy window.

For passive matrix devices (as shown in FIG. 1) strip like row electrodes 6₁ to 6ₘ, e.g. of InSnO₂ are formed on one wall 3 and similar column electrodes 7₁ to 7ₙ are formed on the other wall 4. With m-row electrodes and n-column electrodes this forms an m×n matrix of addressable elements. Each element is formed by the interaction of a row and column electrode. For active matrix devices a discrete nonlinear device e.g. a transistor or diode is associated with each pixel.

For the passive matrix device a row driver supplies voltage to each row electrode 6. Similarly a column driver 9 supplies voltage to each column electrode 7. Control of the applied voltages is from a control logic 10 which receives power from a voltage source 11 and timing from a clock 12.

For an active device e.g. a thin film transistor active matrix liquid crystal device (TFT AMLCD) three types of electrodes are present (pixel, scanning and signal electrodes) as well as a common electrode on the opposite side of the liquid crystal. The control electrode operates the gate such that the voltage on the signal electrode is applied to the relevant pixel electrode.

An example of the use of a mixture and device embodying the present invention will now be described with reference to FIG. 2.

The liquid crystal device consists of two transparent plates, 3 and 4, for example made from glass; in the case of an active matrix device these will usually be of aluminosilicate (alkali free) glass often with a passivation layer of SiO₂. For an active matrix display, the active devices, e.g. thin film transistors, are fabricated and the colour filter layer is added for a full colour display. These plates are coated on their internal face with transparent conducting electrodes 6 and 7, often Indium tin oxide (ITO), which is patterned using photolithography techniques. The transparent plates 3 and 4 are coated with a photoactive sample comprising one or more liquid crystal compounds according to the invention. A typical coating procedure involves the dissolution of one of the compounds of the invention in a solvent, for example cyclopentanone, followed by spin coating of the photoactive compound on the transparent plate. Once the photoactive compound has been coated onto the plates it is exposed to actinic radiation to induce cross-linking of the photoactive molecules. The cross-linking process can be monitored by measuring the birefringence of the alignment layer. The intersections between each column and row electrode form an x, y matrix of addressable elements or pixels. A spacer 5 e.g. of polymethyl methacrylate separates the glass plates 3 and 4 to a suitable distance e.g. 2-7 microns preferably 4-6 microns. Liquid crystal mixture 2 is introduced between glass plates 3, 4 by filling the space between them. This may be done by flow filling the cell using standard techniques. The spacer 5 is sealed with an adhesive in a vacuum using an existing technique. Polarisers 13 may be arranged in front of and behind the cell.

The device may operate in a transmissive or reflective mode (see FIGS. 2 and 3). In the former, light passing through the device, e.g. from a tungsten bulb, is selectively transmitted or blocked to form the desired display. In the reflective mode a mirror, or diffuse reflector (16), is placed behind the second polariser 13 to reflect ambient light back through the cell and two polarisers. By making the mirror partly reflecting, the device may be operated both in a transmissive and reflective mode.

The alignment layers (not shown) have two functions, one to align contacting liquid crystal molecules in a preferred direction, and the other to give a tilt to these molecules—a so called surface tilt—of a few degrees typically around 4° or 5°. In an alternative embodiment, a single polariser and dye mixture may be combined. Liquid crystal compounds of the current invention may also be used in LCDs with an actively addressed matrix e.g. thin film transistors (TFT-LCDs) or a passively addressed matrix e.g., dual scan STN.

1. A liquid crystal compound of Formula (I) with a mesogenic core which comprises at least one group of sub-formula (i) 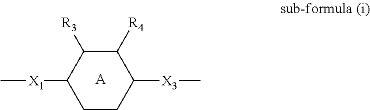
wherein R³ and R⁴ are independently selected from hydrogen, halogen or CF₃, provided at least one of R³ or R⁴ is selected from halogen or CF₃;
A is a 1, 4,-carbocyclic aromatic ring or a fused carbocyclic aromatic ring, which may be optionally substituted;
X¹, X³ are linking groups independently. selected from a direct bond, —S—, —SC(O)—, —(O)——OC(S)—, SC(S)—, —CH₂CH₂—, —(CH₂)₄—, —CH₂O—, —CH═CH—, —≡c—, —COO—, —OCO—, or —OCH₂—, provided that at least one of X¹ or X³, is selected from —S— or —SC(O)—.

2. A compound according to claim 1 wherein R³ and R⁴ are both selected from fluorine.

3. A compound according to claim 1, wherein the group of sub-formula (i) is a group a sub-formula (ii) 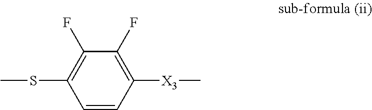
wherein X³ is as defined in claim 1.

4. A compound according to claim 3 wherein X³ is —O— or —S—.

5. A compound according to claim 1, comprising a compound of Formula (II) 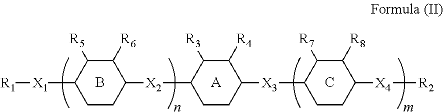
wherein R¹ and R² are independently selected from cyano, halo, a functional group, optionally substituted hydrocarbyl, optionally substituted alkoxy, optionally substituted heterocyclyl, a group R¹³C(O)O— or R¹³OC(O)— where R¹³ is optionally substituted hydrocarbyl;
R³, R⁴ are as defined in claim 1, R⁵, R⁶, R⁷ and R⁸ are independently selected from hydrogen, halogen, cyano or CF₃;
X¹, X³, are as defined in claim 1, X² and X⁴ are independently selected from a direct bond, —S—, —SC(O)——OC(S)—, SC(S)—, —CH₂CH₂—, —(CH₂)₄—, —CH₂O—, —CH═CH—, —C≡C—, —COO—, —COO—, —O— or —OCH₂—;
A is as defined in claim 1, B and C are independentl_{y} selected from carbocyclic aromatic ring, a fused carbocyclic aromatic ring or a heterocyclic ring, any of which may be optionally substituted;
and n is 0, 1 or 2, m is 0 or 1, provided that m+n is 1 or 2, further provided that the at least one —S— or —SC(O)- group is vicinal to at least one halogen or CF₃.

6. A compound according to claim 5, wherein both R¹ and R² are independently selected from optionally substituted alkyl or optionally substituted alkenyl.

7. A compound according to claim 5, wherein, when n=1 and m=1, at least one of X¹ or X² is selected from —S— and both R⁵ and R⁶ are fluorine, or at least one of X² or X³ is selected from —S— and both R³ and R⁴ are fluorine.

8. A compound according to claim 5 wherein X¹, X², X³ andX⁴ are selected from a direct bond, —S—, —(O)—, or —CH₂CH₂—.

9. A compound according to claim 8 wherein at least one of X¹, X², X³ and X⁴ is —S— and at least one is —(O—.

10. A compound according to claim 5 wherein there are two fluorine atoms present on at least one of the rings A, B or C.

11. A compound according to claim 10 wherein at least two of the rings A, B or C each have two fluorine atoms present in the ring.

12. A compound according to claim 5 wherein B and C are independently selected from 1,4-phenylene, 1,4-cyclohexyl, 2,5-dioxanyl, pyridyl or 2,5-pyrimidinyl.

13. A compound according to claim 5 wherein A is 1,4-phenylene or naphthyl.

14. A compound according to claim 5, comprising a compound of Formula (III) 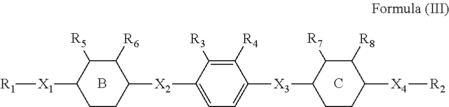
wherein R¹, R², R⁵, R⁶, R⁷, R⁸, X¹, X⁴, B and C are as defined in claim 5, R³ and R⁴ are halogen and at least one of X² or X³ is —S—.

15. A compound according to claim 14 wherein R³ and R⁴ are both fluorine.

16. A compound according to claim 5, comprising a compound of Formula (IV) 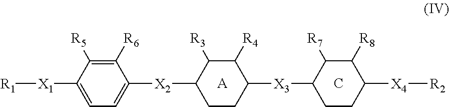
wherein R¹, R², R³, R⁴, R⁷, R⁸, X³, X⁴, A and C are as defined in claim 5, R⁵ and R⁶ are halogen and at least one of X¹ or X² is —S—.

17. A compound according to claim 16 wherein R⁵ and R⁶ are both fluorine.

18. A compound according to claim 5, comprising a compound of Formula (V) 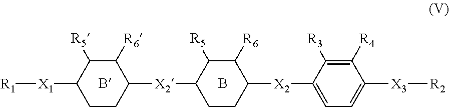
wherein R¹, R², R³, R⁴, X¹, X², X³, B are as defined in claim 5, B′ is selected from B, X²′ is selected from X², R⁵′ and R⁶′ are selected as R⁵ and R⁶ respectively, R³ and R⁴ are halogen and at least one of X³ or X² is —S—.

19. A compound according to claim 18 wherein R³ and R⁴ are both fluorine.

20. A compound according to claim 5, comprising a compound of Formula (VI) 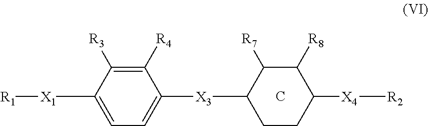
wherein R¹, R², R⁷, R⁸, X⁴, and C are as defined in claim 2, R³ and R⁴ are halogen and at least one of X¹ or X³ is —S—.

21. A compound according to claim 20 wherein R³ and R⁴ are fluorine.

22. A compound of intermediate A 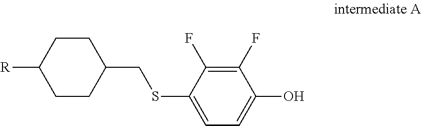
wherein R is a group of R¹ as defined in claim 5.

23. A liquid crystal mixture comprising at least one compound according to claim 1.

24. A liquid crystal device comprising at least one compound according to claim 1.

25. A liquid crystal device according to claim 24 comprising two spaced cell walls each bearing electrode structures and treated on at least one facing surface with an alignment layer, a layer of liquid crystal material being enclosed between the cell walls.

26. A liquid crystal device according claim 24, wherein the device is an Active Matrix Device, an STN device or a TN device.

27-29. (canceled)
